(19) United States
(12) Patent Application Publication
(10) Pub. No.: US 2010/0185107 A1
(43) Pub. Date: Jul. 22, 2010

(54) FLEXIBLY DEFORMABLE CABLE WITH TEXTILE COMPOSITE FOR ELECTROMEDICAL APPLICATIONS

(76) Inventors: Thomas GRASSL, Lubeck (DE); Torge WALL, Lubeck (DE); Thomas GALLUS, Ratekau (DE); Yvo GÄRBER, Kastorf (DE); Christian BULLMANN, Bielefeld (DE); Hans-Ullrich HANSMANN, Barnitz (DE); Hartmut STARK, Stockelsdorf (DE); Frank SATTLER, Lubeck (DE); Henning GERDER, Lubeck (DE)

Correspondence Address: MCGLEW & TUTTLE, PC, P.O. BOX 9227, SCARBOROUGH STATION, SCARBOROUGH, NY, 10510-9227, US

(21) Appl. No.: 12/688,174

(22) Filed: Jan. 15, 2010

(30) Foreign Application Priority Data
Jan. 19, 2009 (DE) 10 2009 005 416.2

(51) Int. Cl. H01B 9/02; H01B 3/30; H01B 7/02; A61B 5/042

(52) U.S. Cl. 600/509; 174/110 R; 174/107; 174/117 R; 600/393

(57) ABSTRACT

A cable for electromedical application and especially for recording the ECG of patients of different body shapes and body sizes is provided including a textile composite (1) formed of a carrier layer (2) and a cover layer (3). At least one electric line (4) is accommodated between the carrier layer (2) and the cover layer (3). After manufacture in a cost-effective manner, the cable offers sufficient protection against short circuits in case of penetration of moisture, on the one hand, and adapts to curved and irregularly shaped structures without problems, on the other hand. The cable textile composite (1) is flexibly deformable without destruction, wherein at least one electric line (4) is electrically insulated by an insulation (5) against the carrier layer (2) and the cover layer (3).

### CROSS REFERENCE TO RELATED APPLICATIONS

This application claims the benefit of priority under 35 U.S.C. §119 of German Patent Application DE 10 2009 005 416.2 filed Jan. 19, 2009, the entire contents of which are incorporated herein by reference.

### FIELD OF THE INVENTION

The present invention pertains to a cable, comprising a textile composite comprising a carrier layer and a cover layer, wherein at least one electric line is accommodated between the carrier layer and the cover layer.

### BACKGROUND OF THE INVENTION

Cables of the type mentioned in the introduction, which are especially suitable for electromedical applications, for example, for use as electrode cables for recording electrocardiograms (ECG), are generally known from the state of the art. The class-forming cables are manufactured, for example, by laminating carrier layers and cover layers in calenders. The carrier layer and the cover layer are often heated here such that these melt at least partially and form a stiff composite after cooling. Such a cable is known, for example, from DE 10 2004 007 875 A1.

However, the class-forming cables are only poorly suitable for use on the human body because they cannot sufficiently adapt to the shapes and sizes of the bodies of the different patients owing to their stiffness. Furthermore, it cannot be ensured in the cables known from the state of the art that an electric line incorporated by lamination between the carrier layer and the cover layer will not establish electrical contacts with the human body when moisture of the body is present.

Even though cables that can be placed on the human body in a functionally correct manner are known, these are often too expensive and must be cleaned after use. The cleaning and possible sterilization of the cable is very complicated especially in clinical practice because it must be ensured that no viruses or microorganisms will remain on the cable. It is necessary precisely in clinical practice to rule out infections in patients. In particular, nosocomial infections must be ruled out.

An electrode cable that can be adapted in terms of length especially by means of folds for ECG measurements is disclosed in U.S. Pat. No. 5,341,806.

### SUMMARY OF THE INVENTION

The basic object of the present invention is to design and perfect a cable of the type mentioned in the introduction such that after cost-effective manufacture, the cable offers sufficient protection against short-circuits in case of penetrating moisture, on the one hand, and adapts without problems to curved and irregularly shaped structures, on the other hand.

According to the invention, a cable is provided comprising a textile composite comprising a carrier layer and a cover layer, wherein at least one electric line is accommodated between the carrier layer and the cover layer. The textile composite lends itself to flexible deformation without destruction. The at least one electric line is electrically insulated against the carrier layer and the cover layer by insulation material.

It was first recognized according to the present invention that a flexibly deformable design of the carrier layer and/or of the cover layer makes it possible to adapt the cable to irregularly shaped structures without generating restoring forces. As a result, the cable is especially suitable for coming into contact with a human body without forming pressure points. It is ensured by the insulation of the electric line against the carrier layer and the cover layer that current is prevented from flowing to the human body even when moisture penetrates into the carrier layer and/or the cover layer. As a result, the cable is especially suitable for being placed on a human body, because sweat or body fluids present cannot bring about any flow of current.

It has been recognized according to the present invention, in particular, that the cable feels like a textile material to the touch and behaves quasi like a textile fabric layer. The cable can be crumpled up, deformed and bent in its entirety like a textile fabric layer.

The cable is precisely not made stiff in its entirety according to the present invention but is flexibly deformable without destruction in its entirety. Furthermore, the cable can be draped in its entirety. This means that the cable is able in its entirety to form folds, to envelope bodies three-dimensionally and to behave in its entirety like a textile material.

The object stated in the introduction is consequently accomplished.

The cable could have a stretchability of up to 15% at least in the longitudinal direction. It is possible as a result to place the cable around elevations and arches and to slightly stretch it. Furthermore, the stretchability makes possible a nondestructive deformation of the cable. It is conceivable against this background that the cable is both elastically stretchable and inelastically stretchable. During inelastic stretching, the cable is brought from an initial length to a final length and the cable essentially retains the final length.

The composite could be made permeable to air at least in some areas by perforations or porous areas. As a result, the cable can be placed directly on a human body without adversely affecting the breathing of the skin A pleasant wearing comfort can be brought about on the human skin by the perforations or porous areas. Therefore, the cable has an especially good breathing activity, because it behaves in its entirely like a textile material and does not cover the skin of the human body tightly.

The carrier layer and/or the cover layer may be connected to one another in a punctiform manner. Areas that have high permeability to air are formed by the punctiform connection. Furthermore, the composite remains soft, does not become stiff and remains flexible. The punctiform connection may be brought about by bonded points or by welded points. The composite shows no or only a very slight permeability to air in the area of the welds or bonds, but areas permeable to air are created between the points. The permeability to air and the stiffness of the composite can be set depending on the selection of the pattern of points or grid.

It is conceivable against this background that the carrier layer and/or the cover layer is designed as a nonwoven. At least the thickness of a nonwoven is variable and a nonwoven is thus compressible because of the porous and loose structure thereof. Furthermore, nonwovens have porous areas of a high permeability to air because of their porous structure. Because of its easy compressibility and high permeability to air, a nonwoven ensures high wearing comfort on the human skin.

It is conceivable against this background that the carrier layer and/or the cover layer is designed as a nonwoven from split fibers, which were manufactured from bicomponent fibers. Bicomponent fibers often have a so-called PIE structure. Such a bicomponent fiber comprises a plurality of elementary fibers, which have a piece-of-cake-like cross section. The elementary fibers are called split fibers and are manufactured by the bicomponent fiber being split into elementary fibers. The splitting may be carried out by water jets. It was surprisingly found that a carrier layer and/or a cover layer, which has split fibers, shows an especially high wearing comfort, high elasticity and high permeability to air. Nonwovens or nonwoven materials of this type are therefore especially suitable for use in the cable being described here.

Besides fabrics or knitted fabrics, nonwovens have proved to be especially advantageous because these can be manufactured in a cost-effective manner. Besides the nonwoven made of split fibers, a nonwoven made of staple fibers or from filaments, i.e., endless fibers, may be used as well, and these fibers do not have to be in the split form.

The composite could be embedded between two enveloping layers. Enveloping layers stabilize the composite and hence the electric line or electric lines, which run within the composite.

The two enveloping layers could be connected to one another in a punctiform manner. Due to the punctiform connection, the cable has high flexibility and deformability or bendability. The stiffness of the cable and the permeability to air thereof can be set depending on the point pattern selected.

At least one electric line could be designed as a conductive paste. A very flat composite can be manufactured due to this concrete embodiment. It is conceivable against this background that the conductive paste has a high elasticity and hence stretchability in the hardened or crosslinked state as well. Such a paste is proposed by PCT/EP2008/007235. The contents of this international patent application (PCT/EP2008/007235) expressly belong to the disclosure of the present specification (the contents of PCT/EP2008/007235 are incorporated herein by reference).

At least one electric line could be designed as a wire. Wires are characterized by high mechanical stability, so that the cable can be wound or bent without problems and made especially in a zigzag, meandering or serpentine shape.

In particular, metallic wires are characterized by high electric conductivity.

It is also conceivable against this background that at least one electric line is designed as a flexible cord. A flexible cord comprises a plurality of metallic wires, which are twisted together. Flexible cords have proved to be especially advantageous because these have high stability and flexibility. The twisted wires can be displaced at least slightly in relation to one another, so that the flexible cord as a whole has high flexibility.

The electric line is surrounded by an electric shielding in a preferred embodiment. It is ensured hereby that the currents or electric signals of the electric line are not interfered with by electromagnetic fields. In particular, a radiation strength and a reradiation characteristic of up to 2.4 GHz according to DIN EN 60601 shall be reached at a limit valve of 3 V/m.

It is conceivable against this background that the electric shielding is designed as an electrically conductive shielding paste. A paste that can be pressed without problems, on the one hand, and can also adapt very well to porous structures, on the other hand, is advantageously used as an electric shielding here. The shielding paste can flow around minor unevennesses on the carrier layer or cover layer in the liquid state and reliably shield the electric line as a result. The shielding paste can, furthermore, flow around and hence enclose the electric line without problems. The above-mentioned conductive paste may be used as an electrically conductive shielding paste. The conductive shielding paste will then advantageously also have high flexibility and stretchability in the hardened or crosslinked state.

The shielding could have a bare wire or a bare flexible cord. The bare wire or bare flexible cord could be placed into the shielding or into the conductive shielding paste or surrounded by same. It is ensured hereby that all parts and areas of the shielding are connected to one another in an electrically conductive manner. High electrical conductivity is important for active shielding. A potential is present on the bare wire in case of active shielding, and the bare wire carries no potential in case of passive shielding.

At least one electric line could run in a zigzag, wavy or meandering pattern. It is specifically conceivable against this background that a wire of a wavy pattern or of a zigzag pattern runs folded with rounded teeth in the composite. It shall be ensured in case of a zigzag shape that the wire not be folded with sharp teeth, but be essentially bent at the corner points. The wire is prevented hereby from being destroyed. An electric line present in a wavy pattern can be pulled to a nearly straight shape by a pulling force, so that the electric line as a whole has high stretchability in the longitudinal direction.

The cable being described here advantageously has a dielectric strength of at least 5 kV between the electric line and a cable surface. Due to this concrete embodiment, the cable is suitable for being brought into contact with the human body. The cable surface now lies directly on the human body. The human body is protected from current flow or an electric shock by the high dielectric strength. A cable of this design can remain on the human body during defibrillation without problems, because its function is not essentially interfered with by the voltages and currents occurring during the defibrillation. The dielectric strength could also be present between the electric lines and the electric shielding and/or the electric lines and the cable surface.

In an alternative embodiment, the cable has electric lines, which run in strands that are or can be separated from one another in at least some areas. The dimension of the cable can be adapted as a result without problems, because each strand can be assigned to another point in space.

The strands could be separated or could be able to be separated from one another by perforations or weakened portions of material in order to adapt the length of the cable. The perforations or weakened portions of material make it possible to tear apart individual areas of the cable in a simple manner.

The cable being described here could have a plurality of electric lines, which run essentially in parallel to one another. It is conceivable that individual lines or a plurality of lines run in strands, which are separated from one another at least in some areas. The separation of the strands in some areas may be brought about by slots, which are formed in the composite or in the enveloping layers. The slots prepared in the composite or in the enveloping layers are designed especially as areas of reduced material thickness. The areas of reduced material thickness could be provided by an ultrasonic welding process. Due to the arrangement of these areas of weakened material, it is possible without problems to tear up the cable at these areas and to create individual strands, which are separated from one another as a result. The cable can be adapted in length hereby to different geometries and dimensions. In particular, it is conceivable that individual areas of the cable are separated from one another in order to run in different directions in space, whereas contiguous areas together run in one direction in space.

It is conceivable against this background that individual areas of the cable can be connected to one another by buttons or Velcro fasteners to form loops in the cable. This connection may be severed when needed in order to bring about lengthening of the cable. The cable may also be compressed in a wave form, quasi like a festoon, in which case it can be stretched along a guide wire for lengthening.

The folded cable may also be held together by ultrasonic spot welding, for example, at the edge of the cable. If this connection is carried out layer by layer, the cable length can be set very practically in a stepwise manner.

The cable being described here could have, for example, especially five electric lines, which are divided into two strands. Two lines would run in a first strand and three lines in a second strand. The strands could possibly be able to be bent and stretched. A cable, which has at least three ends, can be prepared by this concrete embodiment. Such a cable can be placed on a surface, especially an arched surface, and detect or transmit electric signals at different points of the surface. The aforementioned cable may be placed, in particular, on a human body in order to record an electrocardiogram (ECG). One end of the cable could be connected to a device for recording an electrocardiogram, and the other two ends could be connected to electrodes, which lie on the human body. The cable being described here has an elastic stretchability of up to 15% in the longitudinal direction.

In a special embodiment, the cable is additionally folded such that the overall length of the stretched composite is 15% to 50% greater than the length of the composite in the folded, unstretched state.

The cable is preferably free from silicone, latex or PVC. Due to this concrete embodiment, it is especially suitable for being placed directly on the human skin.

The cable could have a fatigue strength under reversed bending stresses of at least 10,000 cycles. A cable of this strength can be used in medical practice without problems, because it meets the mechanical requirements imposed there.

A cable of the type being described here is especially suitable for use as a disposable cable in an arrangement for recording an electrocardiogram. The cable being described here is characterized by high flexibility. In particular, the cable may have a stretchability of up to 15% in the longitudinal direction. This stretchability may be both elastic and inelastic. Furthermore, the cable being described here can be manufactured in a cost-effective manner and can therefore be disposed of after a single-time use. Complicated cleaning of the cable to prevent infection of the patient becomes unnecessary. The insulation of the electric line against the carrier layer and the cover layer makes the cable insensitive to moisture. The reversible compressibility of the carrier layer and/or of the cover layer leads to high wearing comfort. The cable is therefore especially suitable for being placed on the human body.

The cable being described here is preferably plasma-coated. Plasma coating brings about water- or moisture-repellant finish of the cable while the breathing activity thereof is preserved. In particular, fluorocarbons, hydrocarbons or siloxanes may be used as precursors for plasma coating. It is, of course, also conceivable to use mixtures of said substances as a precursor. These substances have proved to be especially suitable for imparting a hydrophobic finish to the cable and for nevertheless preserving a high breathing activity.

There are various possibilities of embodiment and varying the teaching of the present invention in an advantageous manner. Reference shall be made for this to the subclaims, on the one hand, and to the following explanation of the preferred exemplary embodiments of the teaching according to the present invention on the basis of the drawings, on the other hand.

Generally known embodiments and variants of the teaching will also be explained in connection with the explanation of the preferred exemplary embodiments on the basis of the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic exploded view of a cable according to the invention in which the electric line is designed as a flexible cord;

FIG. 2 is a schematic exploded view of a cable according to the invention in which the electric line is designed as a wire; and

FIG. 3 is a schematic exploded view of a cable according to the invention in which the composite is prepared by weld points which are prepared by an ultrasonic welding process.

### DESCRIPTION OF THE PREFERRED EMBODIMENTS

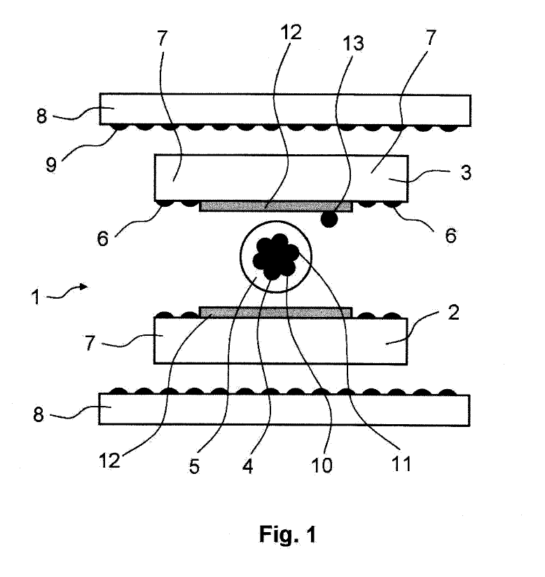

Referring to the drawings in particular, FIG. 1 shows a cable, comprising a textile composite 1 comprising a carrier layer 2 and a cover layer 3, wherein at least one electric line 4 is accommodated between the carrier layer 2 and the cover layer 3 and wherein the textile composite 1 is flexibly deformable without destruction. The electric line 4 is electrically insulated against the carrier layer 2 and the cover layer 3. The carrier layer 2 and the cover layer 3 are reversibly compressible. The cable according to FIG. 1 is characterized by elastic stretchability in the longitudinal direction, i.e., in the direction of the line 4. The carrier layer 2 and the cover layer 3 are connected to one another in a punctiform manner by bonded points. The carrier layer 2 and the cover layer 3 are designed as nonwovens. The nonwovens consist of split fibers, which were produced from bicomponent fibers. In particular, nonwovens of the type with the trade name EVOLON with a basis weight of 60 glm2 were used. Composite 1 therefore has porous areas, which are made permeable to air. The porous areas 7 are located at least between the bonded points 6.

The composite 1 is embedded between two enveloping layers 8. The two enveloping layers 8 are likewise connected to one another and to the composite 1 by means of bonded points 9.

The electric line 4 is designed as a flexible cord 10. The flexible cord 10 comprises a plurality of individual wires 11, which are twisted together. The electric line 4, which is electrically insulated by the insulation 5, is surrounded by an electric shielding 12. The electric shielding 12 is designed especially as a conductive shielding paste. A bare wire 13 is embedded in the conductive shielding paste. The electric shielding 12 is directly in contact with the electric line 4 and is applied to the sides of the carrier layer 2 or cover layer 3, which face the electric line 4.

The carrier layer 2 and the cover layer 3 may also be designed as a foam, which shows sufficient compressibility and elastic stretchability in the longitudinal direction. The same applies to the enveloping layers 8. It is also conceivable to manufacture the enveloping layers 8 from a nonwoven, from which the carrier layer 2 or the cover layer 3 is manufactured as well.

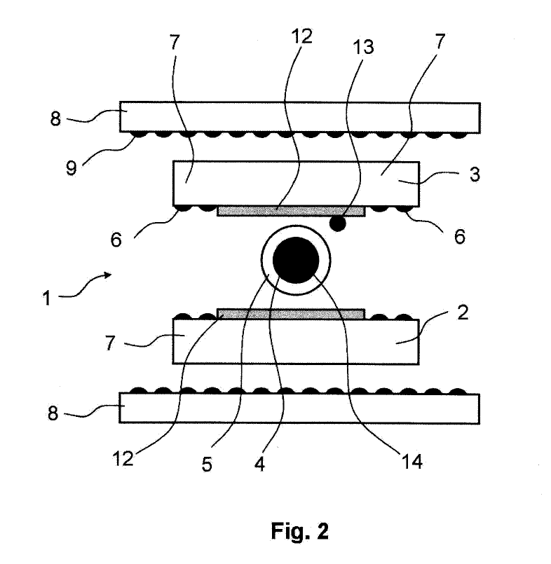

FIG. 2 shows a cable with a design analogous to that of the cable according to FIG. 1, wherein an individual wire 14, which is surrounded by an insulation 5, is used as the electric line 4. The wire 14 is protected from moisture as a result. The cable according to FIG. 2 otherwise has the same design as the cable according to FIG. 1. To avoid repetitions, it shall be pointed out that identical reference numbers in FIGS. 1 and 2 designate identical components of the cable.

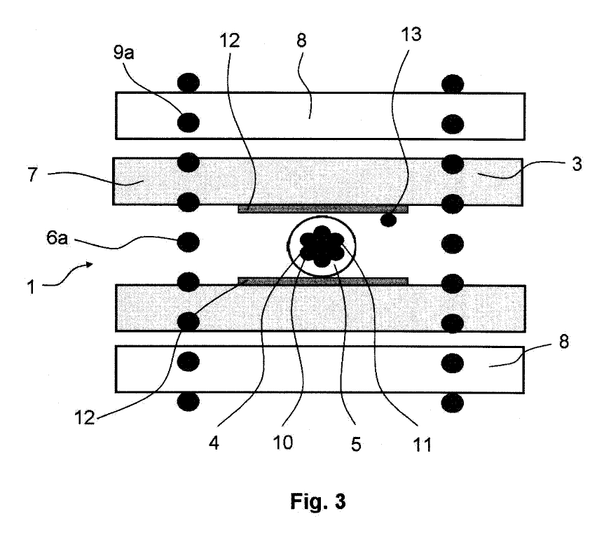

FIG. 3 shows a cable, comprising a textile composite 1 comprising a carrier layer 2 and a cover layer 3, wherein at least one electric line 4 is accommodated between the carrier layer 2 and the cover layer 3 and wherein the textile composite 1 is flexibly deformable without destruction. The electric line 4 is electrically insulated by an insulation 5 against the carrier layer 2 and the cover layer 3. The carrier layer 2 and the cover layer 3 are reversibly compressible. The cable according to FIG. 3 is characterized by elastic stretchability in the longitudinal direction. The carrier layer 2 and the cover layer 3 are connected to one another by weld points 6a in a punctiform manner. The weld points 6a were prepared by ultrasonic welding. The carrier layer 2 and the cover layer 3 are designed as nonwovens. The nonwovens consist of split fibers, which were prepared from bicomponent fibers. Very concretely, nonwovens of the type of, e.g., the trade name EVOLON with a basis weight of 60 glm2 were used. Composite 1 therefore has porous areas, which are permeable to air. The porous areas 7 are located at least between the weld points.

The composite 1 is embedded between two enveloping layers 8. The two enveloping layers 8 are likewise connected to one another and to the textile composite 1 by means of weld points 9a in a punctiform manner. The weld points 9a are prepared by ultrasonic welding.

The electric line 4 is designed especially as a flexible cord 10. The flexible cord 10 comprises a plurality of individual wires 11, which are twisted together. The electric line 4, which is electrically insulated by the insulation 5, is surrounded by an electric shielding 12. The electric shielding 12 is preferably designed as a conductive shielding paste. A bare wire 13 is embedded in the conductive shielding paste. The electric shielding 12 is directly in contact with the electric line 4 and is arranged on the sides of the carrier layer 2 and cover layer 3 that face the electric line 4.

The exemplary embodiments shown in FIGS. 1 through 3 show cables that are characterized by high elasticity and flexibility due to the electric lines 4 used, the electrically conductive shielding paste used as well as the nonwovens used. Very concretely, each cable shows an elastic stretchability of up to 15% of the initial length in the longitudinal direction. For example, a nonwoven of the type of EVOLON from Freudenberg Vliesstoffe KG, 69469 Weinheim, DE, was used as the nonwoven for the carrier layer 2, the cover layer 3 as well as the enveloping layers 8. The nonwovens had a basis weight of 60 glm2. If an individual wire 14 was used as the electric line 4, this was placed into the composite 1 in a meandering or wavy shape in order to ensure the elasticity and longitudinal stretchability of the cable in the entirety thereof. An electrically conductive shielding paste, as is proposed in PCT/EP2008/007235, was used for the electric shielding 12.

In addition to the elastic stretchability of the cable, adaptation of the length of the cable to different body sizes and body shapes can be achieved by means of a folded structure of the composite 1, for example, for premature and newborn babies, on the one hand, and for large and robust adult patients, on the other hand. The length of the stretched composite 1 can thus be greater by, for example, 15% to 50% than in the folded, unstretched state.

Concerning further advantageous embodiments and variants of the teaching according to the present invention, reference is made to the general part of the specification, on the one hand, and to the attached, on the other hand.

Finally, it shall be emphasized in particular that the exemplary embodiments selected previously are used only to describe the teaching according to the present invention, but the teaching is not limited to these exemplary embodiments.

While specific embodiments of the invention have been described in detail to illustrate the application of the principles of the invention, it will be understood that the invention may be embodied otherwise without departing from such principles.

What is claimed is:

1. A cable comprising:
a textile composite comprising a carrier layer and a cover layer;
an electric line accommodated between the carrier layer and the cover layer; and
insulation for insulating said electric line electrically with respect to said carrier layer and said cover layer, wherein said textile composite is flexibly deformable without destruction thereof.

2. A cable in accordance with claim 1, wherein the cable has a stretchability of 0.1% to 15% in a longitudinal direction.

3. A cable in accordance with claim 1, wherein said textile composite is made permeable to air at least in some areas by perforations or porous areas.

4. A cable in accordance with claim 1, wherein one of said carrier layer and said cover layer are connected to the other of said carrier layer and said cover layer in a punctiform manner.

5. A cable in accordance with claim 1, wherein one of said carrier layer and said cover layer comprises a nonwoven.

6. A cable in accordance with claim 5, wherein at least one of said carrier layer and said cover layer is prepared as a nonwoven from split fibers, said split fibers being produced from bicomponent fibers.

7. A cable in accordance with claim 1, further comprising two enveloping layers wherein said composite is embedded between said two enveloping layers.

8. A cable in accordance with claim 7, wherein said two enveloping layers are connected to one another in a punctiform manner.

9. A cable in accordance with claim 1, wherein said electric line comprises a conductive paste.

10. A cable in accordance with claim 1, wherein said electric line comprises a wire.

11. A cable in accordance with claim 1, wherein said electric line comprises a flexible cord.

12. A cable in accordance with claim 1, further comprising electric shielding wherein said electric line is surrounded by said electric shielding.

13. A cable in accordance with claim 12, wherein said shielding comprises an electrically conductive shielding paste.

14. A cable in accordance with claim 12, wherein said shielding comprises a bare wire.

15. A cable in accordance with claim 1, wherein said electric line runs in one of a zigzag, wavy and meandering pattern.

16. A cable in accordance with claim 1, wherein the cable has a dielectric strength of at least 5 kV between said electric line and a surface of the cable.

17. A cable in accordance with claim 1, further comprising at least another electric line to provide a plurality of electric lines that run in strands separated or separable from one another at least in some areas.

18. A cable in accordance with claim 17, wherein said strands are or can be separated from one another by perforations or weakened areas of material in order to adapt the length of the cable, wherein the cable is folded in layers and fixed by weld points.

19. A cable in accordance with claim 17, wherein said plurality of electric lines comprise five electric lines and two strands, wherein two electric lines run in a first strand and three electric lines run in a second strand and wherein said two strands are formed to be bent and stretched in relation to one another.

20. A cable in accordance with claim 1, wherein the cable is silicone-free, latex-free or PVC-free.

21. A cable in accordance with claim 1, wherein the cable has a fatigue strength under reversed bending stresses of at least 10,000 cycles.

22. A cable in accordance with claim 1, wherein the textile composite is folded such that an overall length of the composite in the stretched state is 15% to 50% greater than a length of the composite in the folded, unstretched sate.

23. A method comprising:
providing cable comprising a textile composite comprising a carrier layer and a cover layer, an electric line accommodated between the carrier layer and the cover layer, and insulation for insulating said electric line electrically with respect to said carrier layer and said cover layer, wherein said textile composite is flexibly deformable without destruction thereof;
recording an electrocardiogram using said cable; and
disposing of said cable as a disposable cable following recording an electrocardiogram using said cable.
